(19) United States
(12) Patent Application Publication
(10) Pub. No.: US 2015/0265407 A1
(43) Pub. Date: Sep. 24, 2015

(54) Multi-Layer Distraction Membrane for Bone Defects

(71) Applicant: CELGEN AG, Zug (CH)

(72) Inventor: Domonkos HORVATH, Jestetten (DE)

(21) Appl. No.: 14/731,532

(22) Filed: Jun. 5, 2015

Related U.S. Application Data
(63) Continuation of application No. PCT/EP2013/075638, filed on Dec. 5, 2013.

(30) Foreign Application Priority Data
Dec. 5, 2012 (DE) 102012024206.9

(51) Int. Cl. A61F 2/28

(52) U.S. Cl. CPC A61F 2/2846; A61F 2/2803

(57) ABSTRACT

An artificial bone fragment in the form of a multi-layer membrane for bone formation, in particular a distraction membrane suitable for callus distraction is provided. In addition, the use of the artificial bone fragment for bone regeneration, in particular by callus distraction, and methods for bone regeneration, in particular callus distractions are provided. The distraction membrane includes at least a first, a second and a third layer. The first layer includes collagen. The second layer is rigid and the third layer includes a mineral material. A mating surface is formed by the first layer and a contact surface is formed by the third layer.

### CROSS REFERENCE TO RELATED APPLICATIONS

This application is a continuation of International Application No. PCT/EP2013/075638, filed on Dec. 5, 2013, which claims priority under 35 U.S.C. §119 to Application No. DE 102012024206.9 filed on Dec. 5, 2012, the entire contents of which are hereby incorporated by reference.

### FIELD OF THE INVENTION

The present invention relates to an artificial bone fragment in the form of a multilayer membrane for bone formation, in particular to a distraction membrane suitable for callus distraction, the use of the artificial bone fragment for bone regeneration, in particular by callus distraction, and methods for bone regeneration, in particular callus distraction.

### BACKGROUND

Bone loss is nowadays generally filled with bone replacement materials or autogenous or allogeneic bone.

From a biological perspective, the best replacement material for bone is an autologous spongiosa graft. Such grafts are available only on a limited basis, however, and they exhibit a high absorption rate after transplantation.

The materials and techniques used in the prior art often provide insufficient bone quality, so that implants are not firmly anchored in their implant beds, for example.

The bone replacement material is often used in the form of blocks. The bone replacement is often insufficiently vascularized as a result of the thickness of the blocks, whereby the risk of infection increases, and regeneration is impaired. Growth factors are also frequently used in the methods of the prior art, which greatly increases the cost of the procedures.

Covering membranes, which can be multilayered, are also used to some extent to cover bone defects. These membranes are only intended as a cover. The membranes are flexible and elastic. They can therefore not be moved, in particular not with a controlled movement, during bone formation, for example, during bone distraction by a distraction membrane. The membranes are made of either soft or nonwoven-like material. The membranes are partially impermeable and serve to prevent an ingrowth of connective tissue and blood vessels into the bone defect.

The missing bone substance can be filled in to some extent by bone regeneration instead of using a bone replacement. Segmental interruptions of bone continuity of long hollow bones can thus be treated by distraction osteogenesis.

Callus distraction has been known for more than a hundred years. The most important biological stimulus for bone formation is mechanical stress. Piezoelectric forces that activate osteoblasts and osteoclasts are released in this way. Distraction osteogenesis induces bone formation by triggering biological growth stimuli by slow separation of bone segments. The direct formation of woven bone is achieved by distraction. The defined tensile stress is considerable during bone generation. If such a defined tensile stress is applied to bone fragments, the mesenchymal tissue in the gap and at the adjacent fragment ends displays an osteogenic potential. If there is sufficient vascular potency, then under progressive distraction there occurs a metaplasia of the organized hematoma, also called a blood clot, in a zone of longitudinally arranged fibrous tissue, which can be converted directly into woven bone under optimal external and internal conditions. Complications arise, however, because the bone tissue is subject to highly complex control during its regeneration.

Methods in which bone fragments in the form of previously separated bone blocks are moved away from a bone defect to generate new bone material, as in WO 03/051220 A2, are also applied during distraction osteogenesis. This requires a further operation step. The bone fragment must furthermore be either taken from another bone, for example, the pelvic bone, or the bone defect itself, wherein the bone defect must then not be excessively large, since additional bone must be removed before bone regeneration can take place.

DE 10 2010 055 432 A1, WO 01/91663 A1 and U.S. Pat. No. 5,980,252 describe devices and methods for callus distraction using artificial interfaces, for example, membranes. The membranes are flat or curved plates or discs that are generally made from metal, for example, titanium. These membranes are not biodegradable and thus must either remain in the body of the patient or be removed by an additional operation. The structure, composition, and material of these membranes are also not ideal for good vascularization through the surrounding tissue.

### SUMMARY

According to an aspect of the present invention, a bone replacement material is provided that makes it possible to carry out a bone regeneration procedure that overcomes the disadvantages of the prior art, and in particular that permits a good nutrient supply through the surrounding tissue.

According to another aspect of the present invention, membranes are provided, in particular distraction membranes, which allow better bone regeneration by distraction. The tissue adjacent to the membrane, for example, can be better vascularized than with the membranes from the prior art.

According to a further aspect of the present invention alternatives are provided to the bone fragments separated from the bones, which were used in the prior art.

The technical problem is solved by providing artificial bone fragments in the form of membranes, in particular in the form of distraction membranes.

According to another aspect of the present invention, a membrane, in particular a distraction membrane is provided, for bone formation, preferably as artificial bone fragment for bone formation. The membrane has at least two layers, a contact surface and a mating surface. The mating surface is formed by the first layer, the first layer consists of or largely contains collagen, and the second layer is rigid.

According to another aspect of the present invention, a membrane is provided for bone formation with a contact surface and a mating surface. The membrane has at least two layers, the mating surface is formed by the first layer, the first layer is composed of or largely contains collagen, and the second layer is composed of or largely contains bioresorbable plastic.

In a preferred embodiment, the membrane of the invention is resorbable, in particular bioresorbable.

In one example embodiment the membrane can be made up of two layers. The contact surface is then formed by the second layer.

In a preferred embodiment the membrane is made up of at least three layers.

In a preferred embodiment the membrane is made up of three layers. In a preferred embodiment the membrane is made up of three layers. The contact surface is formed by a third layer.

In a preferred embodiment, the membrane is made up of at least three layers, wherein the contact surface is formed by a third layer, and the third layer consists of or largely contains mineral material. In a preferred embodiment, the membrane is made up of three layers, wherein the contact surface is formed by a third layer and the third layer consists of or largely contains mineral material.

The membrane can thus be made up of two or three layers. The membrane can also be made up of still further layers, such as a fourth layer, a fifth layer, or further layers, for example.

The layer that forms the contact surface, thus in particular the second layer or the third layer, is preferably arranged in such a way that the contact surface allows particularly good adhesion of cells, especially osteoblasts. This is particularly advantageous for a distraction membrane, since this ensures that the adhering cells of the callus are distracted by the movement of the membrane, and thus experience biomechanical pulses.

“Membrane” is understood to be a medical membrane that is used in medical procedures for bone regeneration or for the introduction of bone graft materials into a bone defect. The term bone regeneration is understood in this case to mean both the formation of new bone, for example, by distraction, as well as the introduction and ingrowth of bone replacement materials in a bone defect.

In particular, the term bone regeneration is also understood to mean the regeneration of bone defects, for example, following a cystectomy, tumor surgery, or trauma surgery, etc., independently of the topography, and/or in particular also the regeneration of small bone defects that occur as a consequence of periodontitis, for example.

Membrane is also understood to mean a body that is not curved, that is, a planar or level body. The membrane has a contact surface that serves for attachment or adhesion of osteoblasts in the area of a bone defect, and a mating surface lying opposite to the contact surface. These two surfaces can have any shape, for example, round, oval, square, or polygonal. The contact surface and the mating surface of the membrane in flattened state are preferably rectangular or round. When the membrane is not curved, the size of these two surfaces of a rectangular membrane is derived from the length and width of the membrane. The Furthermore the membrane has at least one side surface, in particular four side surfaces, if it is a rectangular membrane. The size of two of the side surfaces is derived from the height and the length of a rectangular membrane, and the size of the two other sides is derived from the height and width of the membrane in flattened condition. The membrane according to an aspect of the invention is as thin as possible, that is, the size of the side surfaces is many times smaller than the size of the contact surface and the height of the membrane is many times smaller than the length and width of the membrane in the case of a rectangular membrane. The thinner the membrane, the better a substance exchange, for example, a blood exchange, can advantageously take place between the tissue resting on the contact surface and the tissue resting on the mating surface.

Preferably, a membrane according to an aspect of the invention serves for bone regeneration in the oral and maxillofacial region, thus in particular for bone regeneration in the jaw. Jaw can mean the maxilla or the mandible.

In a particularly preferred embodiment, the membrane according to an aspect of the invention is a distraction membrane.

A “distraction membrane” is understood to be a medical membrane that can or does serve as an artificial contact surface in a bone distraction. Such a membrane is preferably rigid and resistant to fracture, and in particular has a stable shape. A distraction membrane is preferably configured such that it can be secured to a distraction device, and in a controlled manner using the distraction device at a desired speed continuously or in individual steps can be separated from the bone, for example pulled or pushed away from the bone. A distraction membrane is thus a specific subgroup of medical membranes that a person skilled in the art can readily distinguish from other medical membranes, which serve for simple covering of a bone defect for example.

“Bone distraction” or “callus distraction” is understood to mean a medical procedure for bone regeneration, in which an element is slowly removed from a bone defect, so that an artificial biomechanical pulse, in particular a tensile pulse, is exerted on the cells, in particular osteoblasts, existing in a callus produced between the bone defect and the element. The element can be for example a bone, bone fragment, or an artificial body. The element can especially be a distraction membrane, and in connection with the invention a distraction membrane according to an aspect of the invention.

“Biomechanical pulse” is understood to be a mechanical force transmission, especially the transmission of a tensile force, to a cell, particularly osteoblasts, and the biological processes triggered thereby in the cell.

It was surprisingly discovered that a membrane can be configured in such a way by a multilayer configuration of a membrane, in particular a distraction membrane, that the contact surface as well as the mating surface can interact particularly well with the respective tissue resting on the surfaces. The membrane according to an aspect of the invention thus represents a surprisingly good replacement for autogenous or allogeneic bone blocks. The collagen of the first layer of a membrane according to an aspect of the invention acts at the same time on the connective tissue resting on the mating surface. This collagen layer can serve at the same time as protection for the rigid second layer. The second layer, which preferably consists of or largely contains bioresorbable plastic, serves as basic structure, so that the membrane can be rigid and unbendable, and is thus suitable for use as distraction membrane. A sufficient stability of the membrane is achieved by the preferred use of resorbable plastics, so that the use of metals, for example, titanium, can be omitted. The entire membrane according to an aspect of the invention can thus be advantageously resorbed into the body, despite its sufficient stability, so that the membrane or membrane parts need remain in the body or again surgically removed. The bioresorbable plastic is furthermore well suited as a contact surface for cells, in particular osteoblasts of a callus, or can be readily coated with a third layer. In a preferred embodiment, this contact surface can therefore be especially formed by a third layer, which preferably consists of or largely contains mineral material. This layer corresponds then advantageously to a natural bone layer of autogenous or allogeneic bone.

The use of bioresorbable plastic as a structural layer furthermore has the advantage that this structural layer can have a specific shape, for example, can be curved and/or can have a predetermined porosity, which promotes blood flow and vascularization. The preferred use of a biodegradable plastic as structural layer furthermore has the advantage that this material can be easily cut or otherwise divided, so that the membrane can be cut into the desired shape without great effort.

The artificial bone fragment according to an aspect of the invention can be advantageously configured as a membrane by the selected materials, so that it can thus be very thin despite the multiple layers. This has the advantage that improved blood flow and vascularization can take place through the thin first layer and the thin second layer and also through an optional thin third layer in comparison with the known natural or artificial bone fragment blocks known from the prior art, which must have a certain thickness.

Without wanting to be tied down by theory, the state of the art assumes that an important factor for bone formation is shielding of the bone defect from ingrowing connective tissue. Shielding membranes, which shield the bone defect from the connective tissue and are supposed to prevent ingrowth of the connective tissue into the callus forming in the bone defect, are therefore frequently used in the treatment of bone defects. This shielding is often considered more important than the presence of biomechanical pulses that stimulate osteoblasts to form bone. Surprisingly, it appears that providing biomechanical pulses is far more important than shielding the bone defect from ingrowth of surrounding connective tissue. The membrane according to an aspect of the invention can therefore be preferably perforated. It can thus have pores that extend from the mating surface through the membrane to the contact surface. Through these pores, both the bone defect and the callus located there as well as also the connective tissue separated by the membrane from the bone defect can be well supplied with nutrients and blood through these pores and can thus vascularize particularly well. Since the membrane is thin in comparison with the bone blocks, the pores are correspondingly short, so that a particularly good connection of the tissue resting on the mating surface to the callus adjacent to the contact surface can be obtained. The fact that connective tissue can at the same time grow into the bone defect area surprisingly plays a subordinate role, as long as the osteoblasts in the callus receive enough biomechanical pulses to be stimulated to form bone.

It was also surprisingly discovered that good new bone formation can be achieved not only by artificial biomechanical pulses, as they are generated, for example, by distraction of the membrane, but also by natural biomechanical pulses produced, for example, in the jaw region by biting, speaking and otherwise moving the jaws against one other. It was discovered at the same time that a membrane according to an aspect of the invention can transmit such biomechanical pulses to the callus in a bone defect, especially if it is fixedly connected to the bone that encloses the bone defect. The membrane according to an aspect of the invention can thus not only be used as a distraction membrane for callus distraction, but alternatively also as a simple membrane resting on the bone defect membrane, particularly if this membrane resting on the bone defect is fixedly connected to the bone that encloses the bone defect, for example, by screws. This use is preferred for defects that are delimited by bone material over at least half of the adjacent surface or at least three sides, since the pulse transfer to the membrane is then particularly good.

The use of the membrane according to an aspect of the invention leads in particular also to good vascularization of the newly formed bone tissue, particularly if the membrane is provided with pores that extend through the membrane in an interconnecting manner from the mating surface to the contact surface according to the invention.

In a preferred embodiment, the second layer is consists of or largely contains bioresorbable plastic material. The second layer has pores having a diameter of at least 10 μm, the pores extending from the interface between the second layer and the first layer to the contact surface through the second layer in an interconnecting manner. In a preferred embodiment, the membrane is a bioresorbable distraction membrane.

The mating surface of the membrane according to an aspect of the invention is formed by a first layer that consists of or largely contains collagen. In a preferred embodiment, the first layer contains at least 50% by weight of collagen. The first layer contains more preferably at least 75% by weight of collagen. Particularly preferably, the first layer contains at least 90% by weight, and even more preferably at least 95% by weight of collagen. The first layer can especially be made exclusively of collagen. The collagen can be native collagen or denatured collagen. The collagen may be a collagen selected from the group consisting of collagen types I through XXVIII and mixtures thereof. The collagen is preferably a collagen of type I, since it is a fibrillar collagen that is found in many connective tissues.

A “first layer” is understood to be the layer that forms the mating surface, which consists of or largely contains collagen.

Preferably the first layer, which consists of or largely contains collagen, has a layer thickness of at least 0.1 mm and at most 10.0 mm.

Preferably the thickness of the first layer is at least 0.1 mm. Particularly preferably the layer thickness of the first layer is at least 0.2 mm. The layer thickness of the first layer is preferably at least 0.5 mm. The layer thickness of the first layer is preferably at least 1.0 mm. The layer thickness of the first layer is preferably at least 2.0 mm.

The layer thickness of the first layer is preferably at most 10.0 mm. The layer thickness of the first layer is preferably at most 5 mm. The layer thickness of the first layer is preferably at most 3 mm. The layer thickness of the first layer is preferably at most 2.5 mm.

The layer thickness of the first layer is preferably at least 0.5 mm and at most 2.5 mm.

The “second layer” is understood to be the layer of the membrane according to an aspect of the invention that forms the basic structure of the membrane according to the invention. The first layer and the second layer preferably form a boundary surface, and thus abut against each other. The second layer is preferably rigid. The second layer can have the shape of a plate or frame.

According to an aspect of the invention, the second layer preferably consists of or largely contains at least one bioresorbable plastic or contains predominantly at least one bioresorbable plastic.

According to an aspect of the invention, the second layer preferably consists of or largely contains a bioresorbable plastic.

In a preferred embodiment, the second layer is composed of at least 75% by weight of bioresorbable plastic. The second layer is more preferably composed of at least 95% by weight of a bioresorbable plastic. In a preferred embodiment, the second layer is composed of bioresorbable plastic.

“Resorbable plastic” or “bioresorbable plastic” is understood to be a plastic which is biodegradable and is broken down in the human body, for example a patient. The second layer can contain a single bioresorbable plastic or two or more different bioresorbable plastics. The second layer of a membrane according to an aspect of the invention is preferably composed of a single bioresorbable plastic.

A person skilled in the art is familiar with various bioresorbable plastics. A person skilled in the art can thereby readily select suitable bioresorbable plastics from which the membrane layers can be produced, and which are suitable as a stable and especially rigid structure. Suitable bioresorbable plastics include, for example, polylactic acids, such as polylactide (PLA) and polycaprolactone (PCL).

The bioresorbable plastic is polycaprolactone in a preferred embodiment.

The bioresorbable plastic is polylactide in a preferred embodiment.

In a preferred embodiment, the second layer is configured as a rigid structure and is porous. The second layer is porous in a preferred embodiment. The second layer has interconnecting pores in a preferred embodiment.

The second layer has a layer thickness of at least 0.01 mm to at most 4 cm in a preferred embodiment. The second layer has a layer thickness of at least 0.1 mm to at most 1 cm in a preferred embodiment.

The second layer has a layer thickness of at least 0.1 mm to at most 3.0 mm in a preferred embodiment.

The second layer preferably has a layer thickness of at least 0.01 mm. The second layer preferably has a layer thickness of at least 0.05 mm. The second layer preferably has a layer thickness of at least 0.1 mm. The second layer preferably has a layer thickness of at least 0.2 mm. The second layer preferably has a layer thickness of at least 0.5 mm. The second layer preferably has a layer thickness of at least 1 mm.

The second layer preferably has a layer thickness of at most 4 cm. The second layer preferably has a layer thickness of at most 2.5 cm. The second layer preferably has a layer thickness of at most 1 cm. The second layer preferably has a layer thickness of at most 0.5 cm. The second layer preferably has a layer thickness of at most 3 mm. The second layer preferably has a layer thickness of at most 2.5 mm. The second layer preferably has a layer thickness of at most 2 mm.

The second layer is preferably rigid, so that as a structure it makes possible the use of the membrane as a distraction membrane, or the transmission of biomechanical pulses.

The term “rigid” is understood to mean that the layer or the membrane is so break-resistant, preferably also dimensionally stable, that it does not bend under normally occurring forces, that is, forces that occur during use according to an aspect of the invention. “Rigid” is understood in particular to mean that the membrane does not break under tensile and tractive forces in the magnitude that they appear in callous distraction, and preferably also does not bend, and thus has a sufficient tensile strength.

In an alternative embodiment, the membrane is not rigid, but only largely rigid or somewhat flexible. Such a membrane can be particularly suitable when it is not used as distraction membrane, but is fixedly connected to a bone and serves for the transmission of natural biomechanical pulses.

The second layer is porous in a preferred embodiment.

The membrane has at least one perforation in a preferred embodiment. The membrane is perforated in a preferred embodiment. In a preferred embodiment the membrane has a plurality of holes, in particular pores, which extend through all the layers and are thus interconnecting. The interconnecting pores preferably extend from the contact surface to the mating surface of the membrane. Such holes advantageously allow the penetration of blood at the contact surface and/or the mating surface of the membrane. The blood can then be directed, for example, by capillary forces, through the membrane to the other surface so that good blood perfusion of the callus tissue and/or the connective tissue resting on the membrane is achieved. Vessels can furthermore be formed in the pores, and thus through the membrane, so that good vascularization of the growing bone can thus take place. The preferred pores, according to an aspect of the invention, in particular interconnecting pores with a diameter of, for example, about 1 mm, thus make it possible for capillaries to grow through the membrane, so that a very good blood circulation and immunity is ensured in the area of the newly formed bone. The perforations allow good perfusion of the mucous membrane that covers the membrane and of the regenerated material between the membrane and the bone.

The second layer has pores, in particular interconnecting pores, in a preferred embodiment.

The interconnecting pores must have a minimum size and thus a minimum diameter that allows erythrocytes with a typical diameter of about 7.5 μm to pass through the pores, thus a diameter of very fine blood vessels.

In a preferred embodiment, the pores have a size, that is, a diameter of at least 0.005 mm and at most 1.5 mm. The pores have a size of at least 0.01 mm and at most 1.5 mm in a preferred embodiment.

The pores have a size of at least 0.005 mm in a preferred embodiment. The pores have a size of at least 0.01 mm in a preferred embodiment. The pores preferably have a diameter of at least 0.1 mm. The pores preferably have a diameter of at least 0.3 mm. The pores preferably have a diameter of at least 0.5 mm. The pores preferably have a diameter of about 1 mm. The pores preferably have a diameter of at most 1.5 mm. The pores preferably have a diameter of at most 1.3 mm. The pores preferably have a diameter of at most 1.2 mm.

The pores preferably extend through the membrane. The pores of the mating surface preferably extend through the membrane to the contact surface. The pores are therefore preferably interconnecting, that is, extend from the mating surface up to the contact surface.

The number of pores preferably depends on the size of the membrane. For example, a membrane having a length of about 20 mm and a width of about 10 mm can have about 10 to 20 pores. Such a ratio of the number of the pores to the membrane surface yields an optimum between the total pore surface, which promotes blood circulation, and the adhesion surface for osteoblasts, which adhere to the membrane during the distraction process.

The contact surface can be formed by the second layer or another layer, for example, a third layer. Here the contact surface is preferably configured in such a way that it makes possible the adhesion of cells, especially osteoblasts. The contact surface is preferably rough. The contact surface is preferably porous. The contact area can at the same time have other pores that are not interconnecting and reach only into the membrane, in particular only into the second or third layer, in addition to the preferred interconnecting pores, so that cells, in particular osteoblasts, can adhere particularly well.

A particularly good contact surface can be formed by a third layer, preferably a third layer of mineral material.

The contact surface of the membrane according to an aspect of the invention is preferably formed by the second layer or a third layer. The contact surface of the membrane is preferably formed by a third layer that consists of or largely contains a mineral material.

A person skilled in the art is familiar with differently suitable mineral materials.

The mineral material of the third layer is preferably hydroxylapatite and/or tricalcium phosphate and/or a titanium oxide.

The mineral material of the third layer is preferably hydroxylapatite and/or tricalcium phosphate. The third layer preferably consists of contains hydroxylapatite, and especially contains predominantly hydroxylapatite. In an alternative example embodiment, the third layer is composed of tricalcium phosphate or contains tricalcium phosphate, and in particular contains predominantly tricalcium phosphate. The mineral material of the third layer can alternatively contain hydroxylapatite and tricalcium phosphate. The third layer consists of or contains titanium oxide in a further alternative example embodiment. The titanium oxide is preferably titanium dioxide, for example, rutile.

The third layer is preferably porous. The interconnecting pores extend through the third layer.

The membrane thus has a porous mineral third layer, which constitutes the contact surface of the membrane.

The third layer has a layer thickness of at least 1.0 μm to at most 1 mm in a preferred embodiment.

The layer preferably has a layer thickness of at least 1.0 μm. The third layer preferably has a layer thickness of at least 2.0 μm. The third layer preferably has a layer thickness of at least 5.0 μm. The third layer preferably has a layer thickness of at least 10 μm. The third layer preferably has a layer thickness of at most 1 mm. The third layer preferably has a layer thickness of at most 0.5 mm. The third layer preferably has a layer thickness of at most 0.2 mm.

The preferred membrane thicknesses are obtained in particular from the addition of the preferred thicknesses of the individual layers. The membrane preferably has a thickness of at least 0.2 mm to at most 13 mm. The membrane preferably has a thickness of at least 0.2 mm. The membrane preferably has a thickness of at least 0.5 mm. The membrane preferably has a thickness of at most 13 mm. The membrane preferably has a thickness of at most 10 mm. The membrane preferably has a thickness of at most 5 mm. The membrane preferably has a thickness of at most 3 mm.

The membrane has a hole for affixing a distraction device or a connection element in a preferred embodiment.

The membrane, especially a distraction membrane, has a connection element for connecting or affixing the membrane to a distraction device in a preferred embodiment. In a preferred embodiment, the membrane has a hole, which is particularly preferably provided in the central area of the membrane, and through which a connection element can be inserted or in which a connection element can be screwed. The hole has a thread in a preferred embodiment. The thread can especially be configured through the hole wall provided in the second layer wall. It is thus preferably provided that the membrane has a hole. The second layer especially extends through the bioresorbable plastic of the second layer and the hole in the region of the second layer has a thread. A connection element can be advantageously screwed into this thread. This connection element is preferably a toothed rack, a screw, a screw eyelet or ring bolt for attaching a line or a fixing element for affixing the membrane to an implant.

In one example embodiment, the membrane is attached to at least one connection element, for example, a screw, a screw eyelet or ring bolt, a line or a toothed rack.

In a preferred embodiment, the membrane is suitable for affixing at least one connection element, in particular at least one screw, a screw eyelet or ring bolt, a line, a tooth rack or a connection element to an implant.

In a preferred embodiment, the membrane according to an aspect of the invention has at least one fixing element. In a preferred embodiment, the fixing element is located on the mating surface of the membrane or extends from the mating surface into the membrane as viewed from the mating surface. The at least one fixing element serves to affix the membrane on at least one actuator. A fixing element can be for example a perforation, an eyelet, or a collar. The at least one fixing element is preferably a perforation, in particular a perforation for introducing a screw. A perforation serving as a fixing element can be a round hole, an elongated hole, or an angular hole. The membrane can also have a number of differently shaped perforations.

The edges of the membrane are rounded in a preferred embodiment.

The membrane can be flat or curved. The membrane can be planar and thus flat, especially with smaller membranes.

Alternatively, however, the membrane can also be curved, in particular if it is used in the jaw area. The membrane is curved in a preferred embodiment.

In a preferred embodiment, the membrane is curved in in a U-shape, especially if it is a membrane for use in the jaw area. A U-shaped curved membrane can completely cover a bone defect, especially in the jaw area, thus from above and from both sides.

In a preferred embodiment, at least a portion of the contact surface and the mating surface is curved. The membrane is thus preferably curved over at least a portion of the length or width of the membrane.

The edges between the contact surface and the at least one side surface and/or the mating surface and at least one of the side faces are rounded in a preferred embodiment.

In an alternative example embodiment, the edges formed by two side faces are rounded.

The membrane has rounded edges in a preferred embodiment.

In a likewise preferred alternative embodiment, the membrane is shaped and dimensioned in such a way that it covers at least part of the surface of a jaw bone facing toward the teeth. A flat or curved membrane can thus be provided, which is bent in a horseshoe shape when it is in a planar, that is, a flat condition, and has a length that the membrane can cover a portion of a maxillary ridge. It can be especially provided that the membrane can cover a large part, for example, up to 80% of the maxillary ridge or the entire maxillary ridge. A person skilled in the art, for example, a dental technician, can easily determine the size and shape of the membrane necessary for covering a desired maxillary ridge area. Broader bone defects, for example, bone defects that extend over more than several teeth, and even the entire maxillary ridge can be advantageously treated with membranes shaped in this way.

The membranes can be tailored in shape and size or can be individually adapted to the treated bone defect.

The membrane according to the invention is a membrane for periodontal regeneration in an alternative embodiment. The membrane according to the invention is a membrane for periodontal regeneration by means of distraction in a preferred embodiment. Periodontal regeneration is understood to mean regeneration of the periodontium, thus not only the bone, but also the periodontal ligament, the periodontal membrane, the gingiva, and the papillae, for example, by “guided tissue regeneration” (GTR). In a preferred embodiment, for periodontal regeneration the membrane is made so small that it can also be utilized in the interdental spaces. In a preferred embodiment, the membrane is made very thin for periodontal regeneration. In a preferred embodiment, the membrane is shaped in such a way for periodontal regeneration that it has at least one projection or segment that can be inserted into an interdental space. The membrane for periodontal regeneration is a single-piece, two-piece or multi-piece membrane in a preferred embodiment. In a preferred embodiment, the membrane for periodontal regeneration has at least one fixing element, for example, at least one perforation, for affixing a bone screw.

In a preferred embodiment, the membrane is suitable for use in bone distraction, in particular of a jaw bone. The membrane of a jaw bone serves in particular for use in bone distraction in a preferred embodiment.

In an alternative example embodiment, the membrane is suitable for use for bone generation by natural biomechanical pulses. The membrane is preferably configured in such a way that it can be affixed on a bone. The membrane is preferably affixed on the bone by at least one screw, especially by at least two screws, preferably by at least four screws. The membrane preferably has holes through which the screws can be guided. The membrane preferably has at least one screw, particularly preferably at least two screws or at least four screws, and in particular four screws, with which the membrane can be affixed on a bone.

The screws are therefore bone screws. The screws are preferably composed of a biodegradable material, such as a biodegradable plastic, such as polylactide or polycaprolactone.

In an alternative example embodiment, the membrane is affixed on the bone by at least one nail or pin, in particular at least two nails or pins, preferably at least four nails or pins. The membrane preferably has holes through which nails or pins can be introduced. The membrane preferably has at least one nail or pin, particularly preferably at least two nails or pins or at least four nails or pins, in particular four nails or pins, with which the membrane can be affixed on a bone. The nails are therefore preferably bone nails. The nails or pins are preferably composed of a biodegradable material, for example, a biodegradable plastic, such as polylactide or polycaprolactone.

According to an aspect of the invention, a distraction membrane is provided for use in callus distraction, in particular for building up a jaw bone by distraction.

According to an aspect of the invention, a distraction membrane is provided for use in periodontal regeneration by distraction.

According to another aspect of the invention, a distraction membrane is used for callus distraction, in particular for building up a jaw bone by distraction.

According to an aspect of the invention, a membrane is used for periodontal regeneration by distraction.

According to an aspect of the invention, a membrane is used for bone regeneration by a transmission of natural biomechanical pulses to a callus via the membrane.

According to an aspect of the invention, a distraction device is provided including a membrane according to an aspect of the invention, a fixing device, and a movable connection element that connects the fixing device to the membrane.

According to an aspect of the invention, a kit is provided that includes at least two of the membranes according to an aspect of the invention. According to another aspect of the invention, a kit is provided that includes at least one membrane according to an aspect of the invention, a fixing device, and a connection element for connecting the fixing device to the membrane. The kit preferably includes an instruction manual.

According to an aspect of the invention, methods for callus distraction is provided, in particular for building up a jaw bone by distraction. A membrane according to an aspect of the invention is applied with the contact surface on a bone segment to be regenerated and tension or pressure is exerted on this membrane via a distraction device. Processes in which the membrane according to an aspect of the invention can be used are known, for example, from WO 01/91663 A1 or U.S. Pat. No. 5,980,252, whose contents illustrate the possible uses for the membranes according to an aspect of the invention using the example of membranes of the prior art, and are incorporated into this application.

Without being restricted to the theory, a distance of approx. 1.5 mm between the membrane and the bone is advantageous at the start of the process in such a distraction process.

A distraction is preferably carried out with the membrane according to an aspect of the invention with a distraction speed of at least 0.2 mm per day up to at most 2.5 mm per day, in particular at least 0.5 mm per day up to at most 2 mm per day. The distraction speed is preferably about 1 mm per day.

According to an aspect of the invention, a method is provided for bone regeneration by a transmission of natural biomechanical pulses. A membrane according to an aspect of the invention is affixed on the bone, in particular a jaw bone, which encloses a bone defect. Several membranes according to an aspect of the invention can also be affixed or a membrane according to an aspect of the invention is used, which is U-shaped and thus encloses the bone defect on three sides. The membrane is preferably affixed on the bone via screws. The membrane preferably serves at the same time as a cover for the bone defect. Because the membrane is affixed on the bone and is rigid or only slightly flexible, it transmits to the bone defect the mechanical pressure pulses that are produced during bone movement, for example, as a result of the jawbone movement during biting and speaking, so that the callus forming in the bone defect and the cells located therein, in particular osteoblasts, experience biomechanical pulses. The bone formation is improved in this way.

### BRIEF DESCRIPTION OF THE DRAWINGS

Example embodiments will now be described with reference to the drawings.

FIG. 1 is a cross sectional view of a three-layer membrane according to an example embodiment.

FIG. 2 is a transversal view of the three-layer membrane according to an example embodiment.

FIG. 3 is a cross sectional view of a three-layer membrane with a hole for affixing a screw according to an example embodiment.

### DETAILED DESCRIPTION

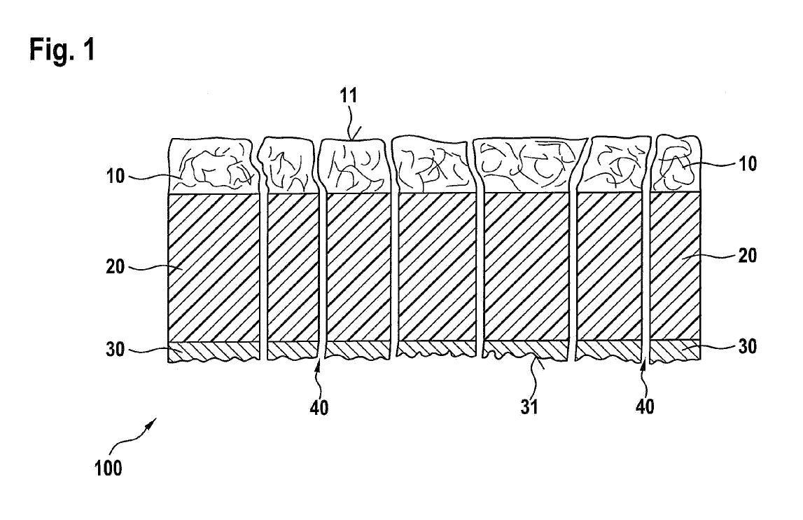

FIG. 1 is a cross sectional view of the three-layer bioresorbable distraction membrane according to an example embodiment. The membrane 100 includes a first layer 10 composed of collagen, a second layer 20 composed of a resorbable plastic, and a third layer 30 composed of a mineral material. The mating surface 11 is formed by the surface of the first layer 10, and the contact surface 31 is formed by the surface of the third layer 30.

The membrane has pores 40, which extend in an interconnecting manner through the membrane from the mating surface 11 to the contact surface 31.

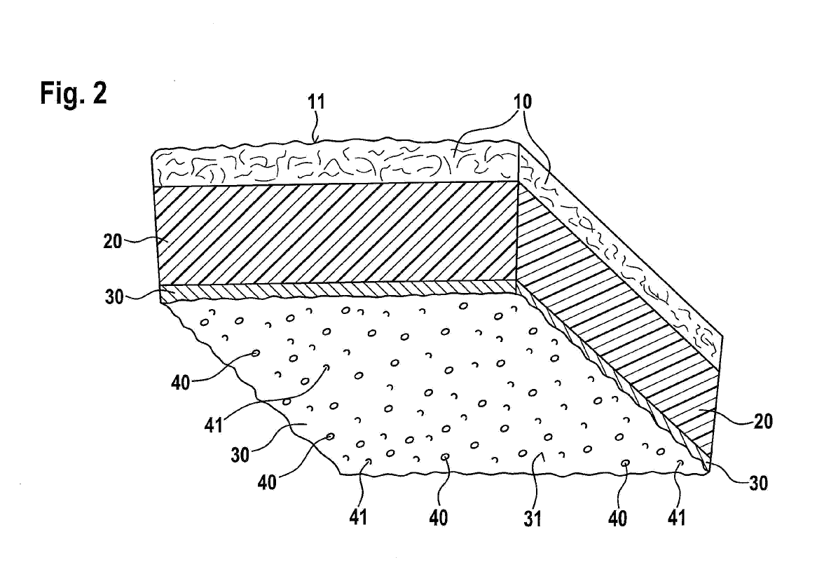

FIG. 2 is a transversal view of the three-layer bioresorbable distraction membrane 100 according to the example embodiment shown of FIG. 1. The contact surface 31, which is composed of mineral material, for example, hydroxylapatite, can be distinguished particularly well. In addition to the interconnecting pores 40, the contact surface 31 has a number of further pores 41. The contact surface is thus porous and thus forms a good contact and adhesion surface for cells, such as osteoblasts.

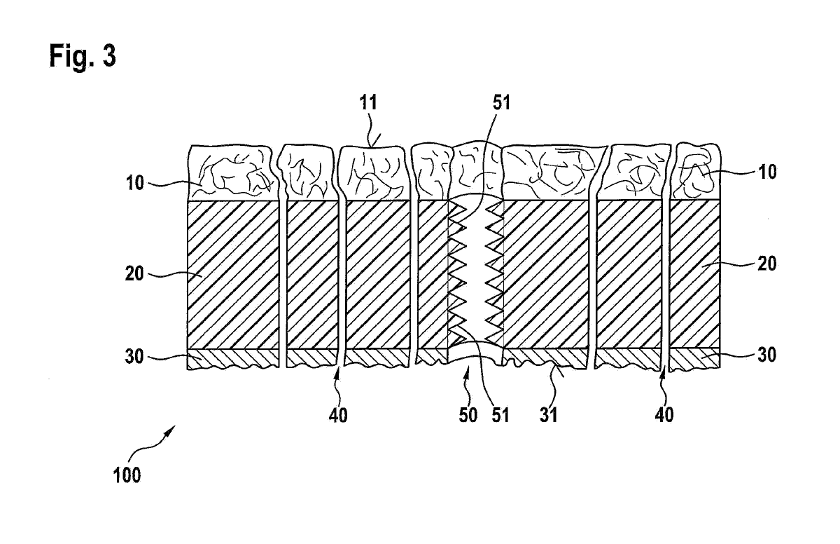

FIG. 3 is a cross sectional view of a three-layer membrane according to an example embodiment. The membrane 100 has a hole 50 in its central region. An inner thread 51 is provided in the area of the second layer 20 composed of resorbable plastic. A screw can thus be screwed into the hole 50, so that the membrane 100 can be connected to a distraction device via the screw and can thus be used as a distraction membrane.

In summary, a distraction membrane for bone formation having a contact surface and a mating surface is provided. The membrane has at least three layers, the mating surface is formed by the first layer, the first layer consists of or largely contains collagen, the second layer is rigid, the contact surface is formed by a third layer, and the third layer consists of or largely contains a mineral material.

According to an example embodiment, the membrane is a bioresorbable distraction membrane, the second layer consists of or largely contains a bioresorbable plastic, the second layer has pores with a diameter of at least 10 μm, and the pores extend through the second layer in an interconnecting manner from the boundary surface between the second layer and the first layer to the contact surface.

According to an example embodiment, the distraction membrane is resorbable.

According to an example embodiment, the second layer consists of or largely contains bioresorbable plastic.

According to an example embodiment, the bioresorbable plastic is polylactide.

According to an example embodiment, the second layer is configured as a rigid structure and is porous.

According to an example embodiment, the distraction membrane has at least one hole for affixing a distraction device or a connection element.

According to an example embodiment, the edges of the distraction membrane are rounded.

According to an example embodiment, the distraction membrane is curved.

According to an example embodiment, the distraction membrane is affixed on at least one connection element, in particular on a screw, a line, or a toothed rack.

According to an example embodiment, the distraction membrane is shaped and dimensioned in such a way that it covers at least a portion of the surface of the jaw bone that faces toward the teeth.

According to an example embodiment, the distraction membrane is provided for use in bone distraction, in particular of a jaw bone.

According to another example embodiment, a distraction device is provided including a distraction membrane according to an example embodiment, a fixing device, and a movable connection element that connects the fixing device to the membrane.

According to an example embodiment, a kit is provided including at least one distraction membrane according to an example embodiment and at least one bone screw or at least one implant or at least one connection element of a distraction device.

What is claimed is:

1. A distraction membrane for bone formation comprising:
at least a first, a second and a third layer, wherein the first layer comprises collagen, wherein the second layer is rigid, and wherein the third layer comprises a mineral material;
a mating surface formed by the first layer; and
a contact surface formed by the third layer.

2. The distraction membrane according to claim 1, further comprising:
a boundary surface between the second layer and the first layer,
wherein the membrane is a bioresorbable distraction membrane, and
wherein the second layer comprises a bioresorbable plastic and has pores with a diameter of at least 10 μm, and the pores extend through the second layer in an interconnecting manner from the boundary surface between the second layer and the first layer to the contact surface.

3. The distraction membrane according to claim 1, wherein the distraction membrane is resorbable.

4. The distraction membrane according to claim 1, wherein the second layer comprises bioresorbable plastic.

5. The distraction membrane according to claim 4, wherein the bioresorbable plastic is polylactide.

6. The distraction membrane according to claim 1, wherein the second layer is porous.

7. The distraction membrane according to claim 1, further comprising at least one hole to affix a distraction device or a connection element.

8. The distraction membrane according to claim 1, wherein edges of the distraction membrane are rounded.

9. The distraction membrane according to claim 1, wherein the distraction membrane has a curved shape.

10. The distraction membrane according to claim 1, wherein the distraction membrane is affixed on at least one connection element, in particular on a screw, a line, or a toothed rack.

11. The distraction membrane according to claim 1, wherein the distraction membrane is shaped and dimensioned to cover at least a portion of a surface of a jaw bone that faces toward teeth.

12. The distraction membrane according to claim 1, configured to be used in bone distraction of a jaw bone.

13. A distraction device, comprising:
a distraction membrane according to claim 1;
a fixing device; and
a movable connection element that connects the fixing device to the distraction membrane.

14. A kit comprising:
at least one distraction membrane according to claim 1; and
at least one bone screw.

15. A kit comprising:
at least one distraction membrane according to claim 1; and
at least one implant.

16. A kit comprising:
at least one distraction membrane according to claim 1; and
at least one connection element of a distraction device.
